SEQUENCE LISTING (11 sequences)

SEQ ID NO: 1 - DNA, 25 bp, Unknown
  Fluorescently labeled oligonucleotide
gtaccaggagtgtttctccagactg

SEQ ID NO: 2 - DNA, 25 bp, M. tuberculosis
ggttgacccacaagcgccgactgtt

SEQ ID NO: 3 - DNA, 26 bp, M. tuberculosis
ggttgacccataagcgccgactgtgt

SEQ ID NO: 4 - DNA, 26 bp, M. tuberculosis
ggttgaccaacaagcgccgactgtgg

SEQ ID NO: 5 - DNA, 25 bp, M. tuberculosis
ggttgacccacaagcgccgactgtc

SEQ ID NO: 6 - DNA, 29 bp, M. tuberculosis
ccagaacaacacgctgtcggggttgacca

SEQ ID NO: 7 - DNA, 29 bp, M. tuberculosis
ccagaacaacacgctgtcggggttgacct

SEQ ID NO: 8 - DNA, 29 bp, M. tuberculosis
ccagaacaacccgctgtcgtggttgaccg

SEQ ID NO: 9 - DNA, 29 bp, M. tuberculosis
ccagaacaacccgctgtcggtgttgaccc

SEQ ID NO: 10 - DNA, 20 bp, M. tuberculosis
ggtcgccgcgatcaaggagt

SEQ ID NO: 11 - DNA, 20 bp, M. tuberculosis
cggcacgctcacgtgacaga

(19) United States
(12) Patent Application Publication
(10) Pub. No.: US 2004/0241713 A1
(43) Pub. Date: Dec. 2, 2004

(54) Composition for immobilization of biological macromolecules in hydrogels, a method for preparing a composition, a biochip, and a method for performing the PCR over biochip

(75) Inventors: Andrei Darievich Mirzabekov, Moscow (RU); Alla Jurievna Rubina, Moscow (RU); Sergei Vasilievich Pankov, Samara (RU); Alexandr Nikolaevich Perov, Moskovskaya (RU); Valentina Vladimirovna Chupeeva, Moscow (RU)

Correspondence Address: LADAS & PARRY, 26 WEST 61ST STREET, NEW YORK, NY, 10023, US

(73) Assignee: INSTITUT MOLEKULYARNOI BIOLOGII IM. V.A. ENGELGARDTA ROSSIISKOI AKADEMII NAUK

(21) Appl. No.: 10/763,949

(22) Filed: Jan. 23, 2004

Related U.S. Application Data
(63) Continuation of application No. PCT/RU01/00445, filed on Oct. 26, 2001.

(30) Foreign Application Priority Data
Jul. 25, 2001 (RU) 2001120905

(51) Int. Cl. C12Q 1/68; C12M 1/34

(52) U.S. Cl. 435/6; 435/287.2

(57) ABSTRACT

The invention relates to molecular biology and bioorrganic chemistry and concerns compositions for immobilising modified oligonucleotides, proteins, nucleic acids and any other molecules carrying unsaturated groups in a hydrogel for producing microchips by means of a photo-induced copolymerisation. Said invention also relates to the production of microchips and a polymerase chain reaction (PCR) on a chip which are used in molecular biology for sequencing and mapping DNA, detecting mutations and for an entire range of medical applications.

### FIELD OF THE INVENTION

The invention refers to the field of molecular biology and bioorganic chemistry and deals with a composition for in hydrogel immobilization of oligonucleotides, proteins, nucleic acids or any other molecules being of biological importance in preparing biochips by method of photo-initiated copolymerization. The invention also refers to the technology for manufacturing biochips having an application in molecular biology for sequencing and mapping of DNA, detection of mutations, and in a whole lot of medical applications.

### BACKGROUND OF THE INVENTION

There are known in the art compositions for immobilization of oligonucleotides and proteins in a polyacrylamide gel in manufacturing biochips by method of copolymerization [1, 2].

[1] F. N. Rehman, M. Audeh, E. S. Abrams, P. W. Hammond, M. Kenney, and T. C. Boles, Nucleic Acids Research, 1999, V.27, No. 15, P. 649-655.

[2] A. V. Vasiliskov, E. N. Timofeev, S. A. Surzhikov, A. L. Drobyshev, V. V. Shick, and A. D. Mirzabekov, BioTechniques, 1999, V.27, P. 592-606.

The compositions being used for manufacturing the biochips cited comprise the following components:

monomers making the basis of the gel being formed;

modified oligonucleotides and proteins bearing the unsaturated groups;

medium for performing the photo-initiated copolymerization.

It is known that acrylamide and N,N′-methylenbisacrylamide are used as a gel forming component and as a cross-linking agent respectively, and their total content being of 10% [1] and 5% [2], and the acrylamide:N,N′-methylenbisacrylamide ratio being equal to 29:1 (C=3.3%) [1] and 19:1 (C=5%) [2].

It is known that modified oligonucleotides and proteins bear methacrylamide [1], acrylamide [2], and allyl [2] groups providing their ability to be copolymerized with gel forming monomers.

It is known that use is made of water-glycerol solutions having ratios of water/glycerol equal to 25:75 [1] and 60:40 [2] to form a hydrogel.

Biochips are known where macromolecules comprising the molecular probe are immobilized in the hydrogel cells fixed on a common substrate and form the regular structure (matrix) [1-5].

[3] Khrapko et al., U.S. Pat. No. 5,552,270;

[4] Ershov et al., U.S. Pat. No. 5,770,721;

[5] Guschin et al., Manual manufacturing of Oligonucleotide, DNA, and Protein Microchips, Analytical Biochemistry, 1997, Vol. 250, No. 2, pp. 203-211.

There are known methods for manufacturing biochips based on hydrogels, in which a technological cycle consists of steps: (1) preparation of substrate, (2) forming a matrix of gel cells on the substrate, (3) application of the solution of biological macromolecules onto cells in line with biochip scheme predetermined, (4) chemical treatment of cells with aim to immobilize molecules of probes, (5) washing and drying of biochips obtained. To form matrix of gel cells, it is known the method of laser ablation of a special light-absorbing layer located under the continuous gel layer having a geometry being complement with respect to the predetermined geometry of cells' body [4], as well as the method of photo-polymerization through a mask [5].

There are also known methods for manufacturing biochips based on a gel in which steps of forming the cells' body and immobilizing molecules of probe are combined together by using a technique of photo- or chemical initiated copolymerization [1, 2]. The essence of these methods consists in that use is made of compositions, which comprise, along with monomer and cross-linking agent, immobilized macromolecules provided with the unsaturated group, which enables an incorporation of these molecules into a polymer net of hydrogel.

It is known a method for manufacturing biochips [1], in which method biochip cells are obtained by polymerization of compositions in droplets supported on a substrate using a micropipette.

It is known a method for manufacturing biochips [2], in which method use is made of a special thin-layer (≈5 μm) chamber having a reaction volume bounded, on the one hand, with a substrate of future biochip and, on the other hand, with a window transparent for UV radiation. The biochip cells have been developed one after another by performing a cycle of the following operations: (1) filling a chamber with composition having a corresponding probe, (2) polymerization of composition by exposure of UV-beam focused into a square blemish of the necessary size in the centre of location of future cell, (3) washing the chamber before its filling with the next solution.

In forming biochips, however, the known compositions for immobilization of biological macromolecules in hydrogels, the biochips, and methods for manufacturing thereof have several drawbacks.

### Drawbacks

1. In manufacturing the gels, the application of acrylamide only, as a gel forming component and N,N′-methylenbisacrylamide as a cross-linking agent, greatly restricts the range of hydrogels obtained by their structure and porosity.

2. Used for manufacturing biochips, the modified oligonucleotides contain the 5′-end methacrylamide group only linked to the oligonucleotide molecule through a phosphamide linkage being labile in acids, or the allyl end group having a low ability to acrylamide and N,N′-methylenbisacrylamide during the copolymerization reaction.

3. The modification of proteins to further immobilization is performed by the two-stage method enabling an insertion of the unsaturated moiety by NH₂-groups only.

4. The known biochips are characterized by low reproducibility of gel cell properties and by heterogeneous distribution of compound being immobilized in the cell volume.

5. In manufacturing biochips, the known method of chemical polymerization of composition ingredients is performed in a damp nitrogen atmosphere that makes worse the efficiency of polymerization.

6. The known method of photo-initiated polymerization applies the wavelength of UV radiation of 254 nm that results in destruction of oligonucleotides.

7. Known methods for manufacturing biochips are technically complicated, and uses are made of procedures not ensuring the necessary uniformity and reproducibility of gel cell properties and are hardly automatized.

The main goal of invention is to develop a biochip ensuring the optimal operation of macromolecules being of biological importance and immobilized therein.

The present invention solves the goal as set out.

### SUMMARY OF THE INVENTION

What is claimed is a composition (K) for polymerization immobilization of biologically important macromolecules in hydrogels on preparation of biochips,

K=aA+bB+cC+dD+eE

comprising

a monomer (A) presenting a substrate in forming a gel and being a derivative of unsaturated carboxylic acids;

a cross-linking agent (B) being a water soluble derivative of unsaturated carboxylic acids;

a modified biologically important compound (C) containing an unsaturated group such as acrylamide having a high affinity with the monomer and cross-linking agent that provide to perform their effective copolymerization;

water medium for performing the photo-initiated polymerization (components D and E), at the same time

as a monomer (A), use is made of acrylamide, methacrylamide, N-[tris(hydroxymethyl)methyl]acrylamide, 2-hydroxyethylmethacrylate, taken separately or together;

as a cross-linkine agent (B), use is made of N,N′-methylenbisacrylamide, N,N′-ethylenbismethacrylamide, N,N′-(1,2-dihydroxyethylene)bisacrylamide, and polyethylene glycol diacrylate, taken separately or together;

initial composition has the total content of monomer and cross-linking agent (a+b) ranging from 3 to 40%, and a monomer to cross-linking agent ratio (a/b) being within a range of 97:3 to 60:40 (3<[b/(a+b)]<40%);

the application of monomers and cross-linking agents as indicated above in various combinations and ratios as above makes it possible to obtain hydrogels having an optimal pore size for operation of immobilized macromolecules being of biological importance;

as an immobilized compound (C) being of biological importance and having the content (c) from 0.0001% to 10%, use is made of either derivatives of oligonucleotides of general formula (I):

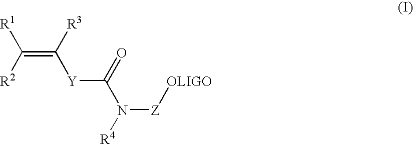

wherein

OLIGO represents an oligonucleotide;

R¹, R², R³ are H, alkyl C₁-C₆, Ph, PhCH₂—;

Z is (CH₂)ₙCH(CH₂OH)CH₂OX where n=1-6; or (CH₂)ₙ—OX where n=2-6;

X is a phosphodiester group binding an unsaturated moiety to 5′- and/or 3′-end of the oligonucleotide;

R⁴ represents H, (CH₂)ₙOH where n=2-6;

Y is (p-C₆H₄)ₙ where n=0-2,

or new derivatives of the DNA of general formula (II)

5′-X—O-DNA-3′-O—Z (II)

wherein

DNA represents a DNA fragment,

X is H or H₂PO₃, Z represents —CO—Y—CR¹═CR²R³

or

X is —CO—Y—CR¹═CR²R³, Z is H or H₂PO₃,

R¹, R², R³ are H, alkyl C₁-C₆, Ph, PhCH₂—;

Y represents (p-C₆H₄)ₙ where n=0-2

or of general formula III

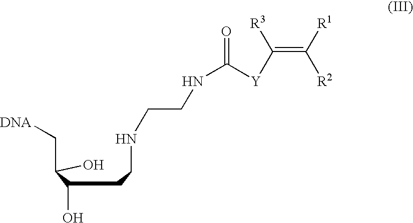

wherein:

DNA represents a DNA fragment;

R¹, R², R³ are H, alkyl C₁-C₆, Ph, PhCH₂—;

Y is (p-C₆H₄)ₙ where n=0-2,

or of general formula (IV)

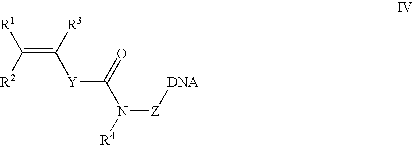

wherein:

DNA represents a DNA fragment;

R¹, R², R³ are H, alkyl C₁-C₆, Ph, PhCH₂—;

Y is (p-C₆H₄)ₙ where n=0-2;

R⁴ represents H, (CH₂)ₙOH where n=2-6;

Z is (CH₂)ₙCH(CH₂OH)CH₂OX where n=1-6; or —(CH₂)ₙ—OX where n=2-6;

X is a phosphodiester group binding an unsaturated moiety to 5′- and/or 3′-end of the DNA fragment,

or new derivatives of the protein of general formula (V)

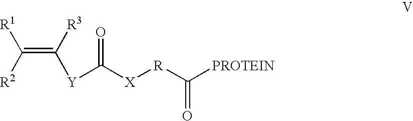

wherein

R¹, R², R³ are H, alkyl C₁-C₆, Ph, PhCH₂—;

X is NH, O, CH₂, S;

Y represents (p-C₆H₄)ₙ where n=0-2;

R is (CH₂)ₙ, (CH₂CH₂O)ₙ, n=1-20,

or of general formula (VI)

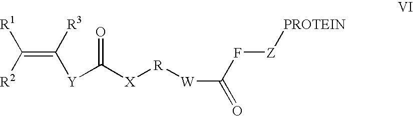

wherein

R¹, R², R³ are H, alkyl C₁-C₆, Ph, PhCH₂—;

X is NH, O, S, CH₂;

Y is (p-C₆H₄)ₙ, where n=0-2;

R is (CH₂)ₙ, (CH₂CH₂O)ₙ, n=1-20;

W is NH, O, CH₂;

F is (CH₂)ₙ, n=1, 2;

Z=NH, S

or of general formula (VII)

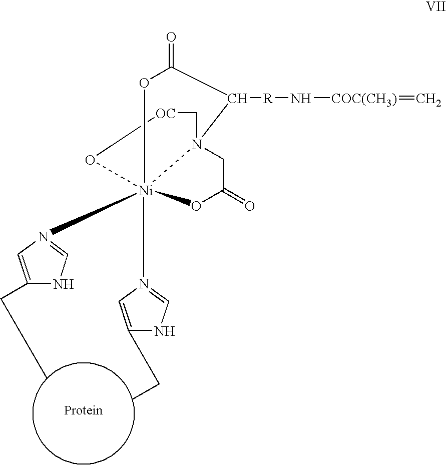

wherein R represents (CH₂)ₙ, (CH₂CH₂O)ₙ, n=1-20;

as a component (D) of water medium in performing a photo-initiated polymerization, use is made of N,N-dimethylformamide and dimethylsulfoxide as a water soluble high-boiling organic compound or glycerol, sucrose, and polyvinyl alcohol as water soluble polyhydric compounds, and the content of aforesaid components (d) being from 0 to 90%.

The invention provides a carrier made of porous glass (CPG) of the following structure:

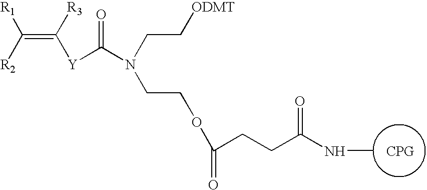

wherein:

R¹, R² are H, alkyl C₁-C₆, Ph, PhCH₂—;

R³ is alkyl C₁-C₆;

Y is (p-C₆H₄)ₙ, where n=0-2,

a method for preparing said carrier by acylation of aminated porous glass,

and method for preparing modified synthetic oligonucleotides of formula I by insertion of fragment of unsaturated acid at 3′- end of oligonucleotide under conditions of automatic solid-phase synthesis using the carrier according to the present invention.

The invention provides methods for preparing the modified DNA fragments

of general formula II—by acylation of DNA fragments with anhydrides of unsaturated acids;

of general formula III—by reductive amination of the purine free DNA followed by acylation of amine derivative with activated esters of unsaturated acids;

of general formula IV—by PCR-amplification using a synthetic primer bearing an unsaturated group at 5′- or 3′- end.

The invention provides methods for preparing the modified proteins

of general formula V—by acylation of protein's free amino-groups with activated esters of unsaturated acids of the following structure:

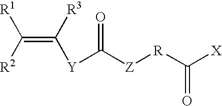

wherein:

R¹, R², R³=H, alkyl C₁-C₆, Ph, PhCH₂—;

Y=(p-C₆H₄)ₙ, where n=0-2;

Z=NH, O, CH₂, S

R=(CH₂)ₙ, (CH₂CH₂O)ₙ, where n=1-20;

X is succinimidoxy-, p-nitrophenoxy-, pentafluoro phenoxy-, or any other readily leaving acceptor group;

of general formula VI by alkylation of protein's amino- or sulfhydryl groups with derivatives of α,β-unsaturated and α-halocarbonyl compounds of the following structure

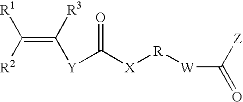

wherein

R¹, R², R³ are H, alkyl C₁-C₆, Ph, PhCH₂—;

X is NH, O, S, CH₂;

Y is (p-C₆H₄)ₙ, where n=0-2;

R is (CH₂)ₙ, (CH₂CH₂O)ₙ, n=1-20;

W is NH, O, CH₂;

Z is a halomethyl, vinyl, or any other fragment comprising an active multiple bond,

of general formula VII by treatment of a recombinant protein comprising an His-6 end fragment with methacrylamide derivatives of nitrilotriacetic acid of general formula

CH₂═C(Me)—CO—NH—R—CH(COOH)—N(CH₂COOH)₂,

wherein R=(CH₂)ₙ, (CH₂CH₂O)ₙ, n=1-20, in the presence of Ni(II) salts.

The invention provides a biochip comprising a substrate pretreated with organosilicon compound, with the purpose of covalent bonding biochip elements to the surface, and discrete gel elements being formed as a result of photo-initiated copolymerization of ingredients of the composition according to the present invention,

at the same time as a organosilicon compound, use is made of 3-trimethoxysilylpropyl methacrylate, 3-trimethoxysilylpropyl methacrylamide, 3-trimethoxysilylpropyl acrylamide, 3-glicidyloxypropyl trimethoxysilane.

The invention also provides a method for manufacturing a biochip, consisting in that the composition according to the present invention is applied as micro droplets onto the substrate treated with aforesaid organosilicon compound, held, and polymerized in an oxygen free inert atmosphere under exposure to UV-radiation (λ≧312 nm), and the biochip obtained is washed from components not reacted during a polymerization.

The application of composition on the substrate is performed by using an automatic device (robot) which is equipped with one or more microdispensers forming a regular structure.

One or more substrates including supported droplets of composition, before and during polymerization, are placed in the sealed container under atmosphere of one of gases (N₂, Ar, or CO₂) with controlled humidity.

The invention also provides a biochip for performing a polymerase chain reaction (PCR) in which use is made of synthetic oligonucleotides (primers) as immobilized macromolecules being of biological importance.

The invention provides a method for performing the PCR on a biochip by

isothermal incubation of biochip with a solution for hybridization, comprising nucleic acid samples under study, to hybridize them with primers immobilized,

isothermal incubation of biochip, comprising nucleic acid being hybridized with immobilized primers, with the amplification solution containing forward (F) and reverse (R) primers,

replacement of the amplification solution out of biochip gel elements with hydrophobic liquid (mineral oil) which completely isolates biochip cells with each other, and

an incubation of biochip under conditions of thermo cycling which provide a realization of PCR-amplification.

### DISCLOSURE OF THE INVENTION

The manufacturing of biochips by using a method of photo-initiated polymerization based on composition of the Invention comprises the following steps:

preparation of composition and substrates for manufacturing a microchip;

application of micro droplets of composition onto a substrate;

holding of micro droplets of composition applied onto a substrate in an inert gas atmosphere;

polymerization in an inert gas atmosphere under exposure to UV-radiation;

washing a biochip obtained.

On preparation of compositions for manufacturing a biochip, all components are carefully mixed to form a homogeneous solution, transferred into plates for micro titration that will be further used as a source of solutions on application onto substrate (substrates) by using an automatic device (robot) which is equipped with one or more microdispensers.

Depending on the content of a water soluble, polyhydric (or high-boiling) component D, there are obtained compositions of various viscosities that allows a size variation of gel elements of biochip under a pin robot diameter fixed. Moreover, ratio of components D in a composition may exert influence on keeping an activity of biologically important compounds being immobilized.

The compositions prepared may be stored at −21° C. at least for 1 year.

As a substrates, there may be used standard preparative glasses, as well as other type substrates, e.g. made of quartz, oxidized silicon, etc.

The preparation of glass to apply a composition for polymerization comprises a cleaning step by a sequential treatment with a concentrated alkali, acid, and a step of chemical modification by using solutions of one of the following organosilicon compound: 3-trimethoxysilylpropyl methacrylate, 3-trimethoxysilylpropyl methacrylamide, 3-trimethoxysilylpropyl acrylamide, 3-glicidyloxypropyl trimethoxysilane in organic solvents.

By modification of glass surface with organosilicon compounds it is possible to obtain biochips which may be used under the pH range from 2 to 12 and temperatures from −10° C. to +100° C.

In biochip, the structure of the cells' body is formed by application of the regular body of micro droplets of polymerization composition onto the substrate prepared. In addition, any micro droplet generally comprises macromolecules of one type. Depending on the viscosity and other properties of the polymerization compositions, and on the desired size of biochip cells, for application, use is made of robots having micro dispensers of various types. In particular, in case of application of viscous solutions having the content of glycerol over 40%, use are made of robot equipped with micro dispensers of a rod (pin) type.

Depending on the rod diameter of dispenser there are obtained droplets (and consequently gel cells) of various size.

Then substrates with micro droplets of composition applied are placed into the sealed container for a period at least 2 h, which container is further filled with dried inert gas (nitrogen, argon, carbon dioxide).

The polymerization process is initiated with UV-radiation (λ≧312 nm) in the inert gas atmosphere. Due to using the wavelength indicated for performing polymerization, a destruction of biologically important macromolecules is eliminated.

The biochips obtained are washed at first in buffer solutions, further in distilled water, and air dried at 25° C.

Various combinations of composition ingredients allow obtaining biochips, which elements' porosity may change over a broad range that makes it possible to use these biochips in many applications, particularly for performing a polymerase chain reaction (PCR), which execution efficiency over the biochip is defined by such factors as:

gel porosity ensuring a facile diffusion of DNA fragments and enzymes,

stability of gel cells over a broad range of pulsating temperatures,

availability and efficiency of immobilized oligonucleotide participation in hybridization and allele specific polymerase reaction to elongate its chain,

as well as to investigate an interaction in systems oligonucleotide-oligonucleotide, DNA-oligonucleotide, protein-protein, protein-DNA, etc.

A biochip as developed for carrying out the polymerase chain reaction and method for realization thereof allow performing the PCR inside the discrete gel elements isolated with hydrophobic liquid (mineral oil) so that each gel element plays a part of micro test-tube.

The present invention is illustrated by the following Examples and Figures wherein:

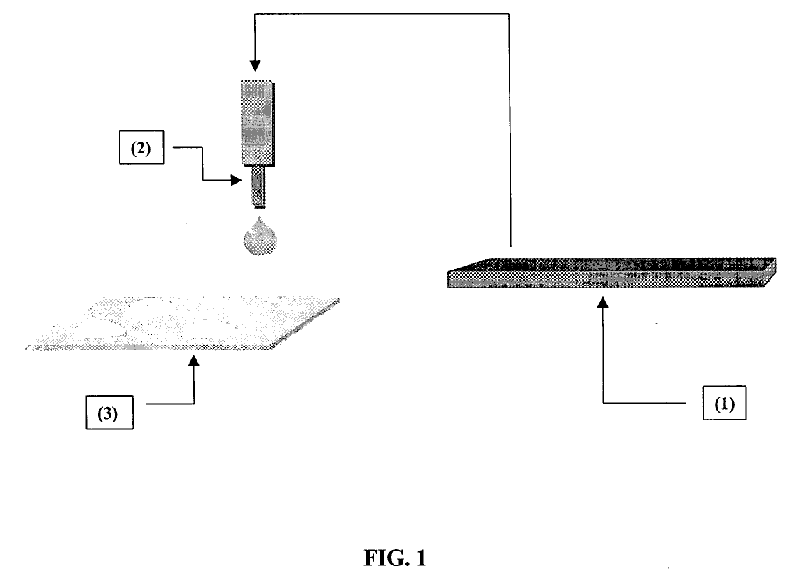

FIG. 1 depicts a process for manufacturing a biochip (Example 1).

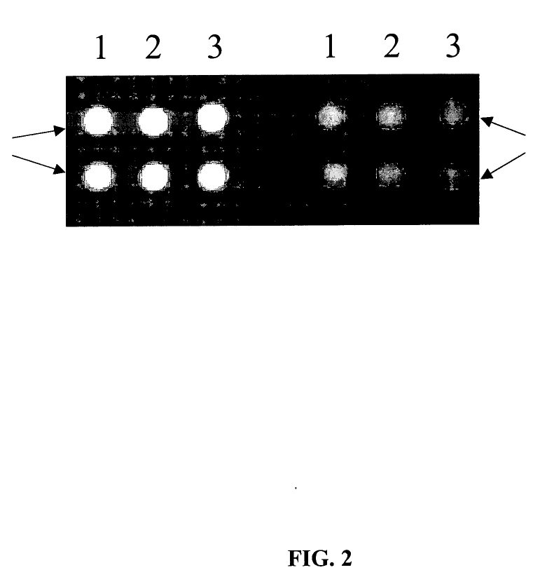

FIG. 2 represents results of hybridization over an oligonucleotide biochip labeled with fluorescent dyestuff “Texas Red”, with immobilized oligonucleotides: entirely complementary and comprising two substitutions inserted by solid-state synthesis with using a modified glass as a carrier and prepared by a procedure described in Example 2. Forming part of compositions No. 1, 2 and 3, oligonucleotides A and B are applied on a glass (by two cells each) and then subjected to polymerization. Fluorescence (bright spots) after the hybridization is observed only in cells of biochip containing the immobilized oligonucleotide A, which is entirely complementary to fluorescent-labeled oligonucleotide.

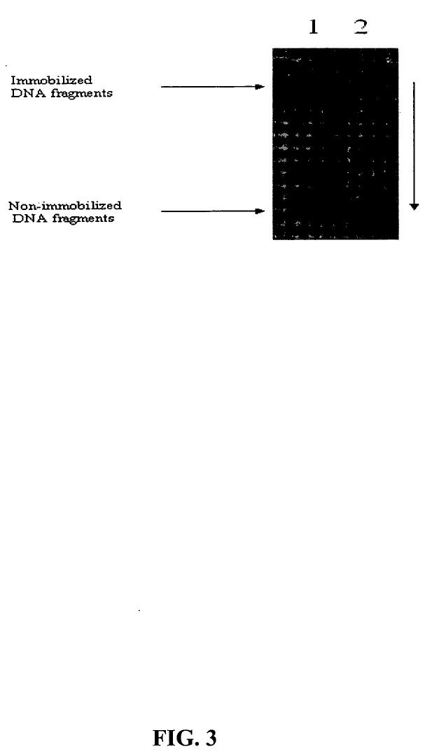

FIG. 3 shows the results of electrophoretic analysis of DNA (human gene fragment ABL, 334 base pairs) after acylation with methacrylic anhydride (Example 3), and copolymerization with acrylamide, and N,N′-methylenbisacrylamide (composition No. 9) (1), and the original DNA (2). Fluorescent picture of electrophoresis is obtained on irradiation (254 nm) of the fluorescent substrate having a polyacrylamide gel.

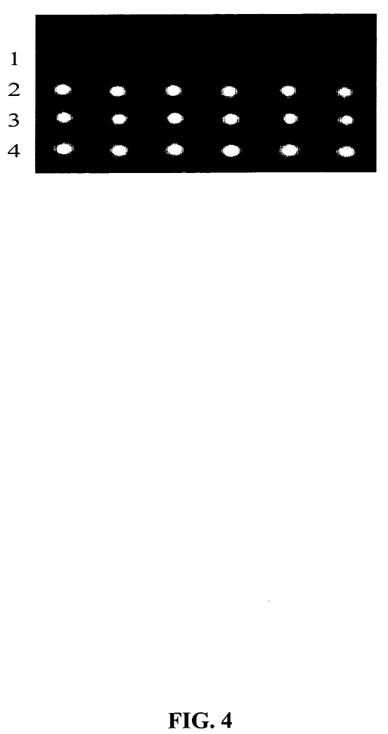

FIG. 4 shows the results of hybridization over the biochip of fluorescent labeled probe with the DNA fragments, which are modified by procedures described in Examples 3-5 and are immobilized in a gel by copolymerization. Row 1 serves as a control which cells are free of the immobilized DNA, since the unmodified DNA fragment forms part of composition. In rows 2-4 (each row includes six identical cells), the cells are obtained by application of composition having modified DNA fragments.

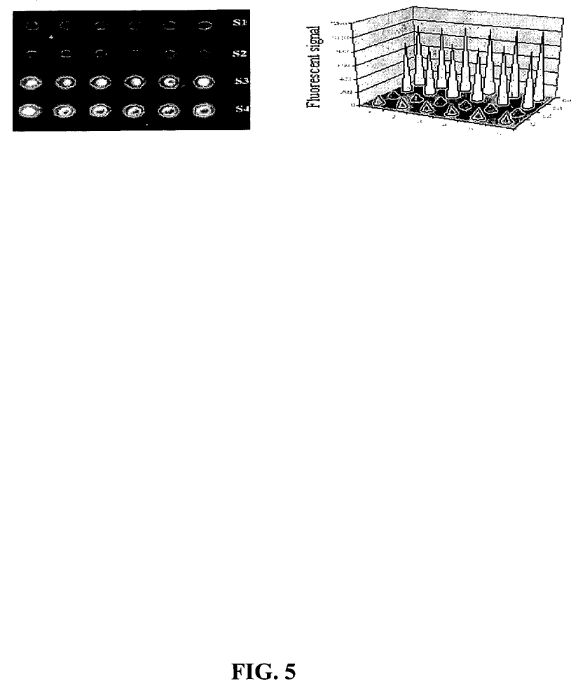

FIG. 5 illustrates the dependence of activity of Barnase protein (immobilized over a biochip) on the extent of its modification (Example 6).

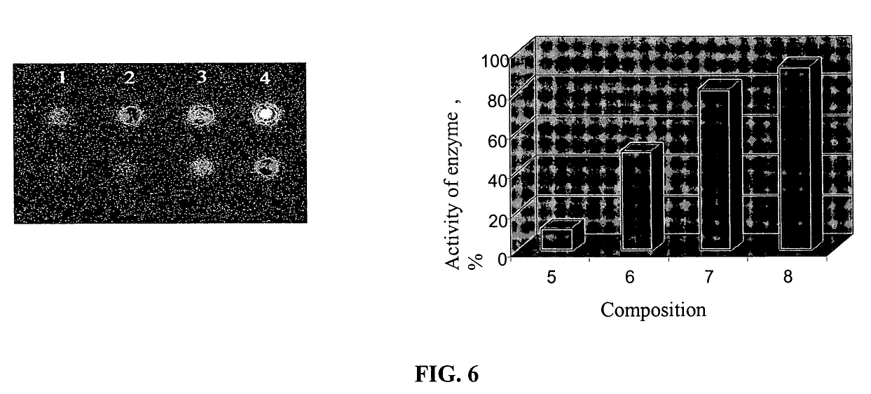

FIG. 6 illustrates the effect of ratio of composition ingredients on conservation of the enzymatic activity of protein being immobilized.

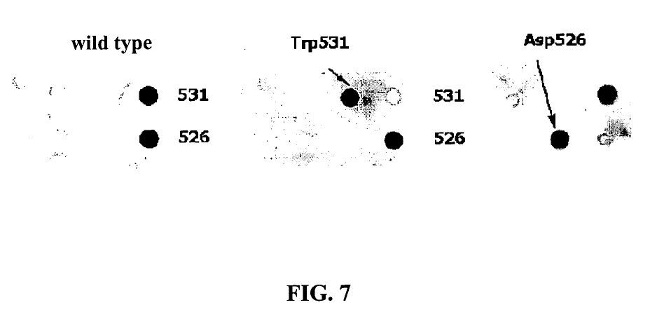

FIG. 7 illustrates the utilization of PCR over biochip wherein amplification takes place both over and inside of gel cell collection of microchip (Example 10).

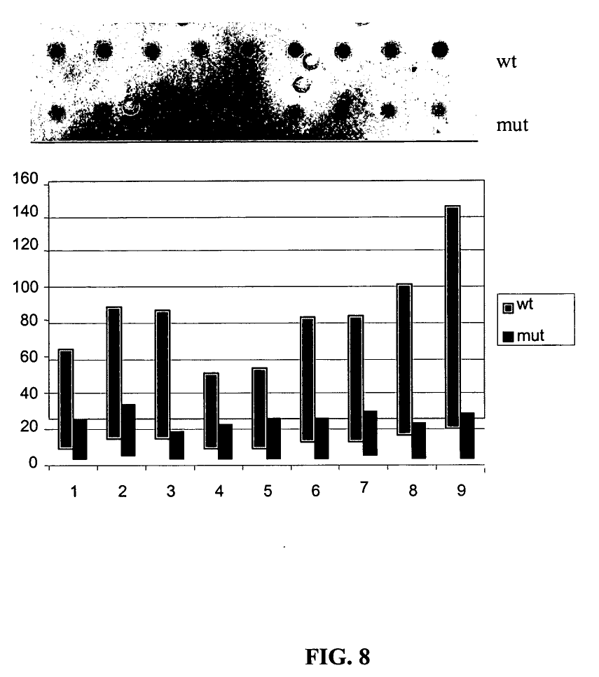

FIG. 8 illustrates the utilization of PCR over biochip wherein amplification takes place inside of gel cell collection surrounded with hydrophobic liquid of gel cells (Example 10).

Table 1 includes data relating to compositions of various mixtures.

### EXAMPLES

1. Manufacturing biochips (Example 1).

I. Manufacturing a composition for gel biochips.

II. Application of composition over substrate and polymerization.

2. Synthesis of methacrylamide-CPG carrier (Example 2).

3. Preparation of the DNA of general formula II- IV (Examples 3-5)

4. Preparation of proteins of general formula V-VII (Examples 6-9)

5. Performing the PCR over biochip (Example 10).

### Example 1

### Manufacturing Biochips

I. Preparation of Composition for Gel Biochips

For manufacturing biochips, uses are made of compositions comprising various ingredients subject to experiment purposes (Table 1). As an example, the manufacturing one of compositions is described.

A solution of modified oligonucleotide or DNA fragment (1-200 picomole/μL in PBS, pH 7.2) or modified protein (0.1-10 mg/mL in PBS, pH 7.2) and glycerol (6.45 μL) is added to methacrylamide (A) and N,N′-methylenbisacrylamide (B) solution in 0.2 M phosphate buffer containing 0.15 M NaCl (PBS), pH 7.2 (1.25 μL, the content of components A and B (a+b)=40% (m/v), ratio a:b=19:1). The mixture is carefully stirred and transferred into plates for micro titration.

II. Application of Composition Over a Substrate and Polymerization Thereof

Preparative glasses (Corning 2947 Micro Slides, Corning Glass Works, Corning, N.Y.) utilized for manufacturing biochips are sequentially treated with an alkali solution, water, sulphuric acid, and water; after drying the glasses are treated with 3-trimethoxysilylpropyl methacrylate (Bind Silane), then wash off an excess of reagent with ethanol, water and air dried. The composition is applied by using a syringe or pin of robot Affymetrix 417 Arrayer (Affymetrix, Santa Clara, Calif.) (FIG. 1). The droplets body obtained is polymerized under exposure to UV-radiation (λ≧312 nm, 40 min, T=37° C.) in a sealed container under an oxygen free inert atmosphere with a controlled humidity. Then biochips are washed in a phosphate saline buffer (0.01M, pH 7.0) comprising 0.1% Tween 20, and in water, and used for performing analysis of various types.

### Example 2

### Synthesis of Methacrylamido-CPG Carrier

Succinic anhydride (0.060 g, 0.3 mmole) and 4-(N,N-dimethylamino)pyridine (DMAP, 0.037 g, 0.3 mmole) are added to solution of N-(2-hydroxyethyl)-N-{2-[di-(4-methoxyphenyl)phenylmethoxy]ethyl}methacrylamide (0.143 g, 0.3 mmole) in dry pyridine (3 mL). The mixture allows standing for 12 h at room temperature. A saturated solution of NaHCO₃ (2 mL) is added to solution. A solvent is evaporated in vacuum. Residuum is dissolved in ethyl acetate (5 mL) and organic layer is sequentially washed with saturated solution of NaHCO₃, several times with water, dried with anhydrous Na₂SO₄ and filtered. A solvent is evaporated and residuum (colorless syrup) is dissolved in dry pyridine (30 mL). To solution is added 2,2′-dipyridyl disulfide (0.060 g, 0.3 mmole), triphenyl phosphine (0.079 g, 0.3 mmole) and the aminated porous glass LCA CPG (1.00 g). A suspension is concentrated in vacuum to a volume of 15 mL and held for 24 h at room temperature under periodically shaking. The suspension is filtered through a glass filter and residuum is sequentially washed with dry pyridine several times. To residuum washed the “capping” solution (acetic anhydride/DMAP/2,6-lutidine/acetonitrile) (10 mL) is added and the mixture allows standing for 5 min. Residuum is sequentially washed with dry pyridine several times, acetone, and diethyl ether. The modified carrier obtained (methacrylamido-CPG) has a concentration of unsaturated groups of 33.7 μmole/g (by test to a trityl group).

FIG. 2 represents the results of hybridization over a biochip having an oligonucleotide labeled with fluorescent dyestuff Texas Red, with immobilized oligonucleotides: entirely complementary and comprising two substitutions inserted by solid-state synthesis with using a modified glass as a carrier and prepared by a procedure above.

### Example 3

### O-Acylation of DNA Fragments with Unsaturated Acid Anhydrides

To solution of methacrylic acid anhydride (15 μL) in N,N-dimethyl formamide (DMF, 100 μL) is added a fragment of DNA (10 picomole) dissolved in H₂O (50 μL) and cooled down to 5° C., then a solution of sodium hydroxide is added by small portions, and the pH-value being maintained of 7. To reaction mixture is added water (500 μL) 15 min later, and the volume of reaction medium is brought to 50 μL by sequential addition of some portions of butanol with following extraction of the aqueous butanol. The modified DNA fragment is precipitated with a solution of lithium perchlorate in acetone (2%, 1000 μL) at −21° C., and a precipitate being centrifuged and washed with acetone.

FIG. 3 represents the result of immobilization performed with copolymerization followed by electrophoresis of the DNA fragment obtained by procedure above. The similar non-acylated DNA fragment serves as a control.

Fragments of DNA are immobilized by method of copolymerization in acrylamide gel (composition No.9, Table 1) as rectangular blocks. The gel with samples is further subjected to electrophoresis for removing the non-immobilized oligonucleotides from gel blocks. The fluorescent picture obtained after performing the electrophoresis shows that DNA fragment (1) containing unsaturated group is completely involved in copolymerization, whereas fragment (2) is nonparticipating in copolymerization.

### Example 4

### Reductive Amination of the Purine Free DNA Followed by Acylation with Activated Ester of Unsaturated Acid

Formic acid (80%, 20 μL) is added to the oligonucleotide or DNA fragment (39.3 nmole) solution in H₂O (15 μL) and held at room temperature for 1 h. The oligonucleotide matter is further precipitated with a solution of lithium perchlorate in acetone (2%, 1000 μL), washed with acetone, and dried. The 0.5M solution of ethylene diamine hydrochloride (50 μL, pH 7.4) is added to the purine free DNA and held for 3 h, at 37° C. Then the makeup solution of sodium hydroboride in water (15 μL, 0.1 M) and 30 min later the solution of ethylene diamine hydrochloride (20%, 14.75 μL) is added. The oligonucleotide matter is precipitated with a solution of lithium perchlorate in acetone (2%, 1000 μL), washed with acetone and dried. Residuum is dissolved in the borate buffer (50 μL, pH 9.3) and the solution of methacrylic acid p-nitrophenyl ester (0.001 g) in DMF (100 μL) is added thereto. The solution is held for 12 h at 35° C. The modified DNA fragment is purified by gel-filtration.

### Example 5

### Preparation of Modified Fragments of DNA by PCR-amplification Using a Synthetic Primer Bearing an Unsaturated Group at 5′- or 3′-end

Fragment DNA (334 base pairs) containing methacrylamide group (MAA) is obtained on PCR-amplification cDNA human gene ABL, cloned to vector pGEM-T Easy Vector (Promega, Madison, Wis.). PCR-reaction is performed by using Taq DNA-polymerase and primers 5′-MAA-Ab15 (direct, F) and A113 (reverse, R). PCR-cycle comprises denaturation (45 s, 94° C.), annealing (60 s, 57° C.), and elongation of chain (45 s, 72° C.). After 35 PCR-cycles, the products are refined by electrophoresis in a gel and dissolved in water.

FIG. 4 demonstrates the results of hybridization over a biochip with immobilized modified DNA fragments being obtained by procedures described in Examples 3-5. Modified DNA fragments being involved in No. 9 composition (Table 1) are utilized for manufacturing a biochip and the hybridization of immobilized DNA is performed with oligonucleotide probe having a fluorescent label. Fluorescence (bright spots) after the hybridization is observed only in cells of biochip containing the immobilized DNA (rows 2, 3, 4). Row 1 serves as a control and comprises gel elements obtained as a result of polymerization of No. 9 composition, which contains the same DNA fragment as obtained on PCR-amplification using unmodified primers.

### Example 6

### Acylation of Proteins at Amine Groups

The procedure of the protein Bamase modification is set forth as an example. A solution of 6-methacryloylaminohexanoic acid ester (20 μL) and N-hydroxysuccinimide (C=0.1 mg/mL) in DMF is added to 100 μL of Bamase solution (C=1 mg/mL) in 0.01 M borate buffer (pH 8.3). A reaction mixture is stirred for 30 min at room temperature. The modified protein obtained is further purified from low molecular weight reaction products and of DMF over micro concentrators Nanosep 3K Omega, washing out the protein with a borate buffer (pH 8.3). In 100 μL of buffer solution, the protein purified has the final concentration of 0.9 mg/mL defined by spectrophotometric analysis. The modified protein obtained comprises one inserted 6-methacryloylaminohexanoic group according to MALDI-MS analysis.

The variation of ratio protein/modifying agent allows manufacturing a protein of a various extent of modification.

FIG. 5 illustrates the dependence of activity of Barnase immobilized over a biochip on the extent of protein modification.

### Example 7

### Alkylation of Proteins at SH Groups

The procedure of alkylation of aspartate-aminotransferase from cytosol of hen's liver with 2-acryloyloxyethyl methacrylate is set forth as an example.

The solution of 2-acryloyloxyethyl methacrylate (0.012 g, 65.2 μmole) in DMF (3 μL) is added to the solution of aspartate-aminotransferase (9 μL, 3.33 mg/mL) in a buffer (25 mM HEPES, pH 8.0, 30 mM KCl, 2 mM MgCl₂) cooled down to 5° C. The homogeneous solution allows standing for 1 h and modified protein is further purified over membrane filters. The effectiveness of protein modification is controlled by an electrophoretic method (similar to that of FIG. 3). Modified protein is utilized for manufacturing protein biochips.

### Example 8

### Synthesis of 2-acryloyloxyethyl Methacrylate

Acryloyl chloride (0.744 g, 8.22 mmole) is added to the solution of 2-hydroxyethyl methacrylate (1.070 g, 8.22 mmole) and triethylamine (0.832 g, 8.22 mmole) in tetrahydrofurane (25 mL) under vigorous stirring. The mixture is stirred at room temperature for 1 h, a precipitate formed is filtered off, and filtrate is concentrated. Water (30 mL) is added to residuum obtained (oil) and the mixture is vigorously shaken. A water layer is separated, ethyl acetate (15 mL) is added, the organic layer is dried over sodium sulfate and solvent is evaporated in vacuum. Yield is of 81%, oil.

¹H NMR (DMSO-d₆) δ: 1.87 (s, 3H, CH₃), 3.34 (m, 2H, O—CH₂), 3.37 (m, 2H, —CH₂—O), 5.68 (pseudo-s, 1H, CH₂═); 6.02 (pseudo-s, 1H, CH₂═); 5.96 (dd, J₁=10.26 Hz, J₂=1.87 Hz, 1H, CH_{2′}═); 6.33 (dd, J₁=17.13 Hz, J₂=1.55 Hz, 1H, CH₂═); 6.19 (dd, J₁=17.13 Hz, J₂=10.28 Hz, 1H, ═CH—). Calculated for C₉H₁₂O₄: C, 58.70; H, 6.52%; Found: C, 58.63; H, 6.64%.

### Example 9

### Synthesis of N-(5-methacryloylamino-1-carboxypentyl)imino Diacetic Acid

To a solution of N-(5-amino-1-carboxypentyl)imino diacetic acid (1.353 g, 5.16 mmole) in water (10 mL), sodium hydrocarbonate (0.563 g, 6.71 mmole) and a solution of 4-nitrophenylmethacrylate (1.069 g, 5.16 mmole) in dimethylformamide (15 mL) are added. Mixture is stirred 12 h at room temperature. Hydrochloric acid is added by small portions to the solution, up to pH 3, cooled down to 5° C. and held for 1 h. The precipitate formed is cold filtered off and filtrate is evaporated in vacuum. Residuum is washed with diethyl ether (3×20 mL) and recrystallization is performed from ethanol. Yield is of 1.210 g (71%), decomposition temperature ˜170° C.

¹H NMR (DMSO-d₆) δ: 1.40-1.45 (m, 2H, CH₂), 1.54-1.59 (m, 2H, CH₂), 1.84 (s, 3H, CH₃), 1.86-1.90 (m, 2H, CH₂), 3,19 (t, J=6.40 Hz, 2H, CH₂), 3.73 (s, 4H, 2CH₂), 3.80 (t, J=6.20 Hz, 1H, CH), 4.84 (s, 4H, NH, 3OH), 5.29 (s, 1H, —CH═); 5.61 (s, 1H, —CH═).

Calculated for C₁₄H₂₂N₂O₇: C, 50.90; H, 6.71; N, 8.48%; Found: C, 50.93; H, 6.80; N, 9.00%.

The compound obtained is utilized for manufacturing a modified protein of formula VII by treatment of the recombinant protein comprising an His-6 end fragment in the presence of Ni(II) salts.

### Example 10

### Polymerase Chain Reaction (PCR) Over a Biochip

Typical PCR-experiment in situ is performed over a biochip in micro-camera. Using the copolymerization method, in different cells of biochip, oligonucleotides are immobilized that completely corresponds to a sequence of wild type, or oligonucleotides comprising an oligonucleotide substitute at 3′-end. Standard PCR-solution (67 mM Tris-HCl, pH 8.6; 2.5 mM MgCl₂; 16.6 mM (NH₄)₂SO₄; 0.001% Triton X-100; 1 mg/mL BSA; 0.24 mM each of dATP, dCTP, dGTP, and dTTP; 2.5 U Taq of DNA-polymerase in 30 μL) also contains about 10⁵ copies of the genomic DNA of Mycobacterium tuberculosis, as well as forward (F) primer (about 1 picomole) and labeled with Texas Red at 5′-end reverse (R) primer (about 10 picomole). Usually, there are performed 35 PCR-cycles: 40 s at 95° C., 60 s at 64° C., and 40 s at 72° C. Once the PCR have finished, the biochip is washed with solution of 0.1-0.3 M NaCl at 80° C. As a result, only duplexes with elongated (due to reaction) primer over the biochip, which may be detected by a fluorescent microscopy.

FIG. 7 illustrates the result of one of these experiments.

### PCR Inside of Gel Elements Under Mineral Oil

Use is initially made of the fragmented (200-300 nucleotides), denaturized genomic DNA of M. tuberculosis for hybridization with the corresponding oligonucleotides over biochip as described above. Then, after washing the DNA not involved in hybridization, the biochip is incubated with the standard PCR-solution (see above) for 30 min at 55° C. Out of gel elements, water solution is substituted with a mineral oil and PCR is performed (30 cycles): 40 s at 72° C., 40 s at 95° C., and 60 s at 64° C. Subsequent to the final step of elongation (10 min at 72° C.), the biochip is carefully washed first with chloroform, then with a solution of 0.1-0.3 M NaCl at 80° C., and is further examined by fluorescent microscopy.

FIG. 8 shows the result of such an experiment. One may see that allele specific PCR again proceeds quite efficiently in cells with a primer being entirely complementary to the DNA used (of wild type, wt, cf. cells with C4 primer). This reaction is notably weaker in cells with a primer comprising an oligonucleotide substitute at 3′-end (mut, C5 primer).

TABLE 1
The compound of the compositions that are used for the production of oligonucleotide, protein and DNA biochips.

| Number | | | | | | | | | | |
|---|---|---|---|---|---|---|---|---|---|---|
| of | | | | | | A content and ratio of the composition | | | | |
| composi- | Ingredients of the composition | | | | | ingredients, % | | | | |
| tion | A | B | C | D | E | a + b | b/(a + b) | c | d | e |
| 1. | methacrylamide | N,N′-methylenebisacrylamide | oligonu- | glycerol | water | 10,000 | 5,000 | 0,033- | 64,500 | 26,467- |
| | | | cleotide | | | | | 0,037 | | 26,463 |
| 2. | acrylamide | N,N′-methylenebisacrylamide | oligonu- | glycerol | water | 5,000 | 5,000 | 0,033- | 64,500 | 30,967- |
| | | | cleotide | | | | | 0,037 | | 30,963 |
| 3. | acrylamide + N- | N,N′- | oligonu- | glycerol | water | 5,000 | 25,000 | 0,033- | 64,500 | 30,967- |
| | [tris(hydroxy- | methylenebisacrylamide + N,N′- | leotide | | | | | 0,037 | | 30,963 |
| | methyl)methyl] | (1,2- | | | | | | | | |
| | acrylamide (1:3) | dihydroxyethylacrylamide))(3:7) | | | | | | | | |
| 4. | methacrylamide | N,N′-methylenebisacrylamide + 2- | protein | glycerol | water | 4,000 | 25,000 | 0,009 | 64,500 | 31,891 |
| | | acryloyloxyethyl methacrylate | | | | | | | | |
| | | (7:3) | | | | | | | | |
| 5. | methacrylamide | N,N′-methylenebisacrylamide | protein | glycerol | water | 5,000 | 7,500 | 0,050 | 60,000 | 35,450 |
| 6. | methacrylamide | N,N′-methylenebisacrylamide | protein | glycerol + | water | 5,000 | 7,500 | 0,050 | 60,000 | 35,450 |
| | | | | sucrose (5:1) | | | | | | |
| 7. | methacrylamide | N,N′-methylenebisacrylamide | protein | glycerol + | water | 5,000 | 7,500 | 0,050 | 60,000 | 35,450 |
| | | | | sucrose (2:1) | | | | | | |
| 8. | methacrylamide | N,N′-methylenebisacrylamide | protein | glycerol + | water | 5,000 | 7,500 | 0,050 | 60,000 | 35,450 |
| | | | | sucrose (1:1) | | | | | | |
| 9. | acrylamide | N,N′-methylenebisacrylamide | DNA | glycerol | water | 5,000 | 5,000 | 5,000 | 64,500 | 26,000 |

1. A composition (K) for immobilization of biological macromolecules in hydrogels by using a copolymerization which have the following formula:
K=aA+bB+cC+dD+eE
wherein:
A is a monomer based on derivatives of acrylic and methacrylic acids;
B is a water soluble cross-linking agent;
C is a biological modified macromolecule bearing an unsaturated group;
D is a water soluble compound as a medium component for performing a copolymerization;
E is water, and
a, b, c, d, e are percentages (X) of each ingredient in the composition (for solids X=m/v×100% and for liquids X=v/v×100%) having the total content of monomer and cross-linking agent ranging from 3 to 40% (3≦(a+b)≦40%), and a monomer to cross-linking agent ratio being within a range of 97:3 to 60:40 and percentages of C, D, and E ingredients being within a range of 0.0001%≦c≦10%; 0%≦d≦90%; 5%≦e≦95%; is useful for production of biochips.

2. The composition of claim 1, comprising acrylamide, methacrylamide, N-[tris(hydroxymethyl)methyl]acrylamide, and 2-hydroxyethylmethacrylate as monomer (A).

3. The composition of claim 1 wherein monomers being used separately or as a mixture.

4. The composition of claim 1 comprising N,N′-methylenbisacrylamide, N,N′-ethylenbismethacrylamide, N,N′-(1,2-dihydroxyethylene)bisacrylamide, and polyethylene glycol diacrylate as cross-linking agent (B).

5. The composition of claim 1 wherein cross-linking agents being used separately or as a mixture.

6. The composition of claim 1 wherein use is made of an oligonucleotide of general formula (I): 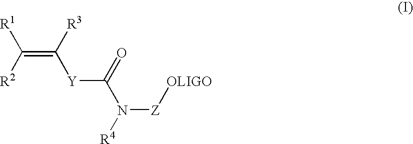
wherein
OLIGO represents an oligonucleotide;
R¹, R², R³ represent H, alkyl C₁-C₆, Ph, PhCH₂—;
Z is (CH₂)ₙCH(CH₂OH)CH₂OX where n=1-6; or (CH₂)ₙ—OX where n=2-6;
X is a phosphodiester group binding an unsaturated moiety to 5′- and/or 3′-end of the oligonucleotide;
R⁴ represents H, (CH₂)ₙOH where n=2-6;
Y is (p-C₆H₄)ₙ where n=0-2,
as a modified biological macromolecule (C) being immobilized.

7. The composition of claim 1 wherein use is made of the DNA of general formula (II)
5′-X—O-DNA-3′-O—Z (II)
wherein
DNA represents a DNA fragment,
X is H or H₂PO₃, Z represents —CO—Y—CR¹═CR²R³
or
X is —CO—Y—CR¹═CR²R³, Z is H or H₂PO₃,
R¹, R², R³ are H, alkyl C₁-C₆, Ph, PhCH₂—;
Y represents (p-C₆H₄)ₙ where n=0-2
as a modified biological macromolecule (C) being immobilized.

8. The composition of claim 1 wherein use is made of the DNA of general formula (III): 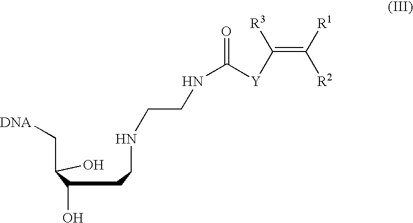
wherein:
DNA represents a DNA fragment;
R¹, R², R³ are H, alkyl C₁-C₆, Ph, PhCH₂—;
Y is (p-C₆H₄)ₙ where n=0-2,
as a modified biological macromolecule (C) being immobilized.

9. The composition of claim 1 wherein use is made of the DNA of general formula (IV) 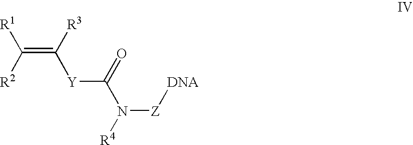
wherein:
DNA represents a DNA fragment;
R¹, R², R³ are H, alkyl C₁-C₆, Ph, PhCH₂—;
Y is (p-C₆H₄)ₙ where n=0-2;
R⁴ represents H, (CH₂)ₙOH where n=2-6;
Z is (CH₂)ₙCH(CH₂OH)CH₂OX where n=1-6; or —(CH₂)ₙ—OX where n=2-6;
X is a phosphodiester group binding an unsaturated moiety to 5′- and/or 3′-end of the DNA fragment,
as a modified biological macromolecule (C) being immobilized.

10. The composition of claim 1 wherein use is made of the protein of general formula (V) 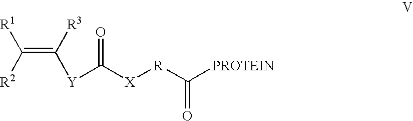
wherein
R¹, R², R³ are H, alkyl C₁-C₆, Ph, PhCH₂—;
X is NH, O, CH₂, S;
Y represents (p-C₆H₄)ₙ where n=0-2;
R is (CH₂)ₙ, (CH₂CH₂O)ₙ, n=1-20,
as a modified biological macromolecule (C) being immobilized.

11. The composition of claim 1 wherein use is made of the protein of general formula (VI) 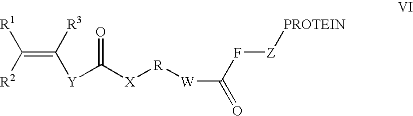
wherein
R¹, R², R³ are H, alkyl C₁-C₆, Ph, PhCH₂—;
X is NH, O, S, CH₂;
Y is (p-C₆H₄)ₙ, where n=0-2;
R is (CH₂)ₙ, (CH₂CH₂O)ₙ, n=1-20;
W is NH, O, CH₂;
F is (CH₂)ₙ, n=1, 2;
Z=NH, S
as a modified biological macromolecule (C) being immobilized.

12. The composition of claim 1 wherein use is made of the protein of general formula (VII) 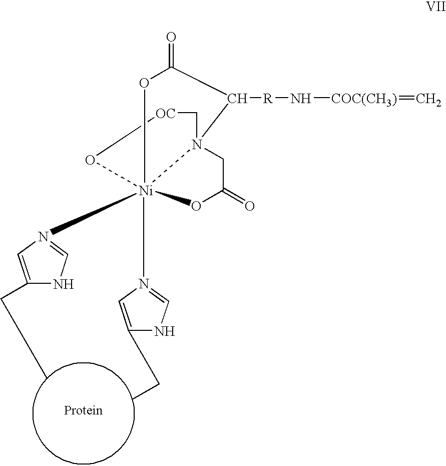
wherein R represents (CH₂)ₙ, (CH₂CH₂O)ₙ, n=1-20;
as a modified biological macromolecule (C) being immobilized.

13. The composition of claim 1 wherein use is made of a water soluble high-boiling organic compound as a component (D) of medium for performing a copolymerization.

14. The composition of claim 13 wherein use is made of N,N-dimethylformamide and dimethylsulfoxide as a water soluble high-boiling organic compound.

15. The composition of claim 1, characterizing in that use is made of a water soluble polyhydric compound as a component of medium for performing the photo initiated polymerization.

16. The composition of claim 15, characterizing in that glycerol, sucrose or polyvinyl alcohol are used as water soluble polyhydric compounds.

17. A method for preparing a composition (K) according to claim 1 for immobilization of biological macromolecules in hydrogels by using a copolymerization consisting in that components of mixture of the following formula:
K=aA+bB+cC+dD+eE
wherein:
A is a monomer based on derivatives of acrylic and methacrylic acids;
B is a water soluble cross-linking agent;
C is a biological modified macromolecule bearing an unsaturated group;
D is a water soluble compound as a medium component for performing a copolymerization;
E is water, and
a, b, c, d, e are percentages (X) of any ingredient in the composition (for solids X=m/v×100% and for liquids X=v/v×100%) having the total content of monomer and cross-linking agent ranging from 3 to 40% (3≦(a+b)≦40%), and a monomer to cross-linking agent ratio being within a range of 97:3 to 60:40 and percentages of C, D, and E ingredients being within a range of 0.0001%≦c≦10%; 0%≦d≦90%; 5%≦e≦95%;
are mixed until the formation of a homogeneous solution which being degassed and being useful for production of biochips.

18. Modified DNA fragments of the following structure:
5′-X—O-DNA-3′-O—Z (II)
wherein
DNA represents a DNA fragment,
X is H or H₂PO₃, Z represents —COY—CR¹═CR²R³
or
X is —CO—Y—CR¹═CR²R³, Z is H or H₂PO₃,
Y represents (p-C₆H₄)ₙ where n=0-2;
being prepared by direct acylation of DNA fragments with anhydrides of unsaturated acids;
or formula (III): 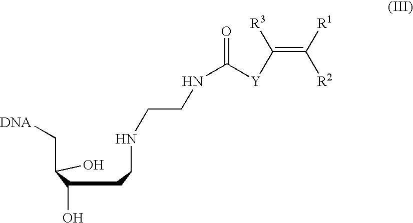
wherein:
DNA represents a DNA fragment;
R¹, R², R³ are H, alkyl C₁-C₆, Ph, PhCH₂—;
Y is (p-C₆H₄)ₙ where n=0-2,
being prepared by reductive amination of the purine free DNA followed by acylation of amine derivative with activated esters of unsaturated acids;
or 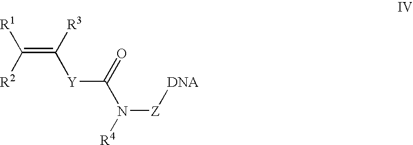
wherein:
DNA represents a DNA fragment;
R¹, R², R³ are H, alkyl C₁-C₆, Ph, PhCH₂—;
Y is (p-C₆H₄)ₙ where n=0-2;
R⁴ represents H, (CH₂)ₙOH where n=2-6;
Z is (CH₂)ₙCH(CH₂OH)CH₂OX where n=1-6; or —(CH₂)ₙ—OX where n=2-6;
X is a phosphodiester group binding an unsaturated moiety to 5′- and/or 3′- end of the DNA fragment,
being prepared by PCR-amplification using a synthetic primer bearing an unsaturated group at 5′- or 3′- end.

19. Modified proteins of the following structure: 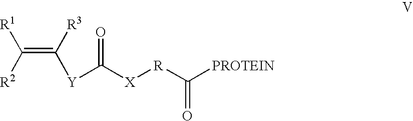
wherein
R¹, R², R³ are H, alkyl C₁-C₆, Ph, PhCH₂—;
X is NH, O, CH₂, S;
Y represents (p-C₆H₄)ₙ, where n=0-2;
R is (CH₂)ₙ, (CH₂CH₂O)ₙ, n=1-20,
being prepared by acylation of protein's free amino-groups with activated esters of unsaturated acids;
or 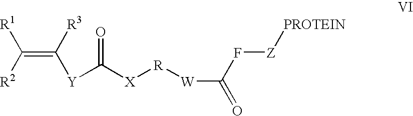
wherein
R¹, R², R³ are H, alkyl C₁-C₆, Ph, PhCH₂—;
X is NH, O, S, CH₂;
Y is (p-C₆H₄)ₙ, where n=0-2;
R is (CH₂)ₙ, (CH₂CH₂O)ₙ, n=1-20;
W is NH, O, CH₂;
F is (CH₂)ₙ, n=1, 2;
Z=NH, S
being prepared by alkylation of protein's amino- or sulfhydryl groups with derivatives of αβ-unsaturated and α-halocarbonyl compounds;
or 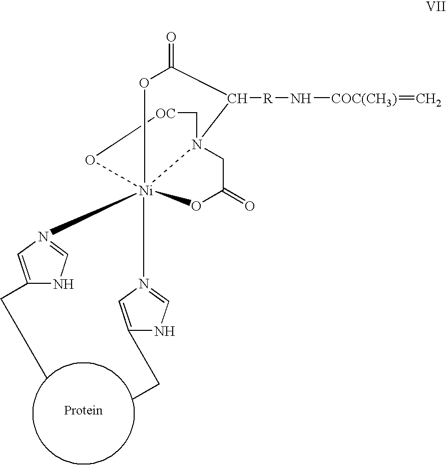
wherein R represents (CH₂)ₙ, (CH₂CH₂O)ₙ, n=1-20;
by treatment of a recombinant protein comprising an His-6 end fragment with methacrylamide derivatives of nitrilotriacetic acid in the presence of Ni(II) salts.

20. A modified porous glass (CPG) of the following structure: 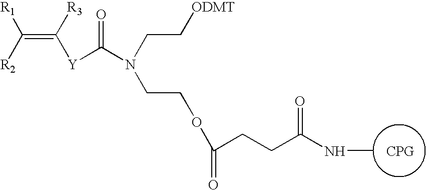
wherein:
R¹, R² are H, alkyl C₁-C₆, Ph, PhCH₂—;
R³ is alkyl C₁-C₆;
Y is (p-C₆H₄)ₙ, where n=0-2,
as a carrier to insert the fragment of unsaturated acid at 3′-end of oligonucleotide of formula I according to claim 6 under conditions of automatic solid-phase synthesis.

21. Activated esters of the following structure: 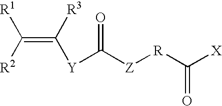
wherein
R¹, R², R³ are H, alkyl C₁-C₆, Ph, PhCH₂—;
Y is (p-C₆H₄)ₙ, where n=0-2;
Z is NH, O, CH₂, S
R is (CH₂)ₙ, (CH₂CH₂O)ₙ, where n=1-20;
X is succinimidoxy-, p-nitrophenoxy-, pentafluoro phenoxy-, or any other readily leaving acceptor group,
as a modifying agent for preparing the protein of formula V.

22. Carbonyl compounds of the following structure: 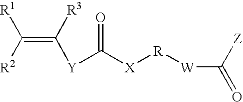
wherein
R¹, R², R³ are H, alkyl C₁-C₆, Ph, PhCH₂—;
Y is (p-C₆H₄)ₙ, n=0-2;
X is NH, O, S, CH₂;
R is (CH₂)ₙ, (CH₂CH₂O)ₙ, n=1-20;
W is NH, O, CH₂,
Z is a halomethyl, vinyl, or any other fragment comprising an active multiple bond, as a modifying agent for preparing the protein of formula VI.

23. Methacrylamide derivatives of nitrilotriacetic acid of general formula:
CH₂═C(Me)CO—NH—R—CH(COOH)—N(CH₂COOH)₂,
wherein R is (CH₂)ₙ, (CH₂CH₂O)ₙ, n=1-20,
as a modifying agent for preparing the protein of formula VII.

24. Biochip accomplished based on composition of claim 1 wherein gel layer formed on a substrate is divided by empty spaces into several cells and each cell will comprise or not comprise macromolecules immobilized, and macromolecule being immobilized in various cells will have different nature and properties.

25. Biochip according to claim 24 wherein said cells form the regular one- or two-dimensional structure (phase).

26. Biochip according to claim 24 on preparation of which an application of the polymerization mixture on substrate is preferably carried out by using an automatic device (robot) equipped with one or more micro dispensers.

27. Biochip according to claim 26 on preparation of which use is made of micro dispensers of rod type.

28. Biochip according to claim 26 on preparation of which use is made of contactless micro dispensers of jet type.

29. Biochip according to claim 26 on preparation of which use is made of several micro dispensers forming a regular structure.

30. Biochip according to claim 24 on preparation of which one or more substrates including applied droplets of polymerization mixture, during polymerization, are placed into a sealed container under oxygen free inert atmosphere with a controlled humidity.

31. Biochip according to claim 24 on preparation of which said container being filled with one of the following gases: N₂, Ar, CO₂.

32. Biochip according to claim 24 on preparation of which said gaseous medium being continuously or periodically restored in the container with substrates.

33. A method for performing the PCR over biochip according to claim 24 by using:
an addition of amplification solution, forward (F) and reverse (R) primers of samples of nucleic acids under investigation, and
an incubation of biochip under conditions of a thermocycling treatment providing a realization of PCR-amplification.

34. A method for performing the PCR over biochip according to claim 24 by using:
an isothermal incubation of biochip with hybridization solution comprising the samples of nucleic acids under investigation to perform their hybridization with primers immobilized (synthetic oligonucleotides),
an isothermal incubation of biochip, comprising the nucleic acids being hybridized with primers immobilized, in the amplification solution containing forward (F) and reverse (R) primers,
replacement of the amplification solution out of biochip gel elements with hydrophobic liquid (mineral oil) which completely isolates biochip cells with each other, and
an incubation of biochip under conditions of a thermocycling treatment providing a realization of PCR-amplification.
